(19) United States
(12) Patent Application Publication
(10) Pub. No.: US 2024/0100330 A1
(43) Pub. Date: Mar. 28, 2024

(54) ELECTRICALLY ASSISTED MUSCLE PUMPING SYSTEM AND METHOD THEREOF

(71) Applicant: NEURONOFF, INC., Cleveland, OH (US)

(72) Inventors: Manfred Franke, Cleveland, OH (US); Stephan Nieuwoudt, Cleveland, OH (US)

(21) Appl. No.: 18/373,910

(22) Filed: Sep. 27, 2023

Related U.S. Application Data
(60) Provisional application No. 63/410,533, filed on Sep. 27, 2022.

(51) Int. Cl. A61N 1/36; A61B 5/00; A61B 5/389; A61N 1/05

(52) U.S. Cl. CPC A61N 1/36003; A61B 5/389; A61B 5/6823; A61B 5/6831; A61N 1/0558; A61N 1/36139; A61N 1/36171; A61B 2562/0219

(57) ABSTRACT

The present disclosure provides an electrically assisted muscle pumping (EAMP) system and a method. The system includes sensors to measure an acceleration of a user. The system includes a processor operatively coupled with the sensors, to register an orientation of the user. Electrodes may be operatively connected to stimulation signal generators to generate electrical stimulation signals. Implanted helical wire structure electrodes are placed inside the user and operatively connected to the electrodes to receive the electrical stimulation signals from the electrodes and transmit the electrical stimulation signals to nerves inside the body of the user. The stimulation signal generators are operatively connected to the processor and adapted to apply electric currents to the electrodes. A recorder is connected to the sensors and the stimulation signal generators to capture the acceleration of the user and the electric currents applied to the electrodes.

### CROSS-REFERENCE TO RELATED APPLICATIONS

This application claims the benefit under 35 U.S.C. § 119(e) of U.S. Provisional Patent Application No. 63/410,533, which was filed Sep. 27, 2022 and titled “ELECTRICALLY ASSISTED MUSCLE PUMPING SYSTEM AND METHOD THEREOF,” which is hereby incorporated herein by reference in its entirety.

### PRIORITY STATEMENT

This application claims priority to, and the full benefit of, United States provisional patent application number 63/410,533 filed on September 27, 2022.

### INCORPORATION OF PRIOR APPLICATION

This application incorporates PCT/US2021/033007 (the '007 application) filed on May 18, 2021, as if set forth entirely herein. The present application uses reference numbers as used in the '007 application.

### FIELD OF THE INVENTION

The present disclosure generally relates to combating Gravity-induced Loss of Consciousness (G-LOC). In particular, the present disclosure provides an electrically assisted muscle pumping (EAMP) system and a method thereof to combat the G-LOC.

The invention herein is an Injectrode system comprising an Injectrode as described in the '007 application and other components, usages, and configurations.

As used herein, an “Injectrode” is the same as, and interchangeable with, a helical wire rope structure.

The invention is a device and a system incorporating a helical wire structure rope electrode as described herein, in the '007 application, and with additional aspects disclosed in the present application. In various embodiments, the Injectrode is configured to be injected into a body near or on a tissue target such as a peripheral nerve, a nerve ganglion, or an organ and be anchored without tines, hooks, or sutures. The Injectrode includes at least one wire rope as an intermediate stage, but the wire rope by itself does not have the properties of the Injectrode which includes a primary wire rope comprising multiple parallel wire strands, with the wire rope having been wrapped helically around a guidewire to form a tertiary wire structure, i.e., a helix comprising coils, and flexible, bendable, bunchable, stretchable, compressible, and pushable. The present invention also includes improvements to various aspects of the Injectrode.

The embodiments of the Injectrode disclosed in the '007 application are fully implanted. One of these embodiments is an injectable device for delivering electrical current to a tissue target in a body. The device is configured for loading into and injection from a dispenser to be fully implanted in the body. The device comprises a helical wire rope structure having an initial length, first and second ends, and coils of at least one twisted wire rope. The twisted wire rope comprises strands of conductive wire configured so that some of the strands can loosen partially from and be spaced apart from the coils. The helical wire rope structure from the first end to the second end having a hollow core, is substantially linear along all of the initial length when loaded inside the dispenser, having capabilities of stretching and flexing, having capabilities of bunching and bending up to 180 degrees upon injection. The helical wire rope structure is configured to allow formation near the tissue target of a first irregular shape near the first end to self-anchor the first end without tines, hooks, or sutures and to provide an electrical charge interface to the tissue target. The helical wire rope structure is surrounded between the first and second ends by a nonconductive coating comprising at least one gap or hole. As further described in the '007 application, in one embodiment, the bunching and the bending of the Injectrode allow formation in a subcutaneous region of a second irregular shape near the second end and the second end and/or a substantially linear portion of the helical wire rope structure are configured to collect electrical current by capacitive and resistive coupling transcutaneously with an external pulse generator (EPG) or stimulation signal generator for generating an electrical waveform. The EPG is the body and transmits current which is transmitted passively along the helical wire rope structure to the tissue target.

Further, this application incorporates PCT/US23/31099 (the '099 application) filed on Aug. 24, 2023, as if set forth entirely herein. The invention herein is an Injectrode system comprising an Injectrode as described in the '099 application and other components, usages, and configurations.

As used herein, the “Injectrode” is the same as, and interchangeable with, a helical wire rope structure. The Injectrode possesses a general shape and pattern of a helix made of at least one wire rope which comprises a number of strands. Although the general shape and pattern of the helix is predictable among all embodiments given standardized manufacturing techniques, the placement of an individual strand and the actual shape and dimension of a single coil in the helix may be random or irregular, but only in this sense within the general shape or the pattern. In the same way, a bunching anchor from a helical structure with a given wire composition and standardized manufacturing may be random or irregular, but only in this sense within a general shape and pattern.

The Injectrode, in various embodiments, comprises conductive wire selected from a group consisting of platinum-iridium, gold, silver, platinum, stainless steel, titanium, titanium-nickel, iridium, tungsten, platinum-tungsten and other metal alloys such as nickel-cobalt base alloy (MP35N), a cobalt-nickel-chromium alloy with molybdenum added for corrosion resistance. Wires comprising the above metals are readily available commercially in the 2-300 micron diameter range, and wires of other diameters are also suitable for some embodiments of the present invention. The final selection of material and diameter for a particular embodiment is dependent on patient biocompatibility and desired tensile (mechanical) and electrical properties for the particular application and embodiment. It is also dependent on the optimum force supplied by the Injectrode onto the tissues against which it is pressed, because mechanical forces due to differences in material compliance or flexibility from any implanted Injectrode influence formation of encapsulation tissue. Wires of biocompatible metals have different mechanical properties, such as hardness, and electrical properties, such as conductivity, and the potential effects for heating of the wires during the conduction of electrical current. The metal composition of the wires can be varied to introduce desired physical properties. The deployment process partially unwinds some of the strands from the main helical structure and pushes them between 1 to 200 microns away that are mechanically distant enough from the helical structure to be mechanically free to move with the tissue and have less encapsulation between that strand and the native tissue. Also, the strands that partially unwind are very flexible (because they are so thin and can move even more easily than a coil of the helical structure) and may be even closer to the tissue target and help with the conduction of energy as the electrical field reduces over distance. In this way the deployment process partially unfolds the tightly wound wire rope forming the helical structure and helps to create more flexibility and thus better mechanical matching between the helix and the surrounding tissue. This is a helical macrostructure with a multi-strand microstructure.

### BACKGROUND

Generally, bodily liquids such as blood and lymph have a tendency to move within a human body when the body is being accelerated. For example, when an airplane pilot performs positive or negative gravity flight maneuvers such as turning left, right, up, or down, the bodily liquids move within the body. A major problem with dynamic changes in acceleration is that the blood may be displaced away from vital organs such as the brain, and instead accumulating in the abdomen and legs of the pilot. As a result, a visual field of the pilot may narrow due to the reduced blood and oxygen supply to the retina. Tunnel vision and grey-out (loss of color vision) are especially applicable for positive gravity. The ability to focus and think may be reduced due to reduced blood and oxygen supply to the cortex of the brain, and is especially applicable for positive gravity. The ability to stay awake is reduced once the blood pressure drops beyond a level that triggers baroreceptors in a neck. A pressure signal is interpreted by a brainstem as a critical level to trigger loss of consciousness, a natural reflex to bring a person into a horizontal position, and re-establish blood pressure to carotid arteries and thereby the brain. Unfortunately, this may kill the pilot due to loss of control over the airplane or an aircraft. This is especially applicable for positive gravity. Under negative gravity, blood pressure of the pilot may increase in the head, running the risk of the dangerous condition known as redout, with too much blood pressure in the head and eyes.

Gravity-induced Loss of Consciousness (G-LOC) is defined as a state of altered perception where one's awareness of reality is absent as a result of sudden, critical reduction of cerebral blood circulation caused by increased gravity force. Due to high level of sensitivity that the eye's retina has to hypoxia, symptoms are usually first experienced visually. As the retinal blood pressure decreases below an intraocular pressure (usually 10-21 mm Hg), blood flow begins to cease to the retina, first affecting perfusion farthest from an optic disc and a central retinal artery with a progression towards a central vision. Skilled pilots may use this loss of vision as their indicator that they are at maximum turn performance without losing consciousness. Recovery is usually prompt following removal of gravity force, but a period of several seconds of disorientation may occur. Absolute incapacitation is a period of time when an aircrew member is physically unconscious and averages about 12 seconds. Relative incapacitation is a period in which the consciousness has been regained, but the person is confused and is unable to perform simple tasks. This period averages about 15 seconds. Upon regaining cerebral blood flow, a G-LOC victim usually experiences myoclonic convulsions (often called as ‘funky chicken’) and often full amnesia of an event is experienced. Brief but vivid dreams have been reported to follow the G-LOC. If the G-LOC occurs at low altitude, this momentary lapse may prove fatal and even highly experienced pilots may pull straight to a G-LOC condition without first perceiving the visual onset warnings.

Blood and to a lesser extent lymph are contained in stretchable vessels within the body. As acceleration is applied to the body as a whole, the blood and the lymph may pool to specific locations within the body that function as a reservoir such as venous vessels for blood, from which it will return back to the heart and thereby be accessible for circulation once an amount of acceleration applied has been reduced.

Further, aircraft pilots, astronauts, racecar drivers, and others expecting to experience sufficient acceleration during their line of work (or pleasure) causing said acceleration forces are trained to rhythmically contract their abdominal and leg muscles. They are trained to tense up their abdomen and their legs (especially lower leg muscles) to utilize a “muscle pump”. The muscle pump forces the blood in a pulsatile fashion back up towards the heart. The muscle pump supplies the heart with sufficient volume that an ejection fraction of the heart does not reduce to a level beyond which insufficient blood volume and/or pressure is being supplied to the neck and head of the person. These repeated pulsatile muscle contractions are to be applied under voluntary and intentional control by the individual in question. In addition to that, the individual is asked to exhale forcefully in a similar rhythmic manner and time the abdominal contractions with the exhaling of air. This helps with a better engagement of the abdominal muscles and increases intra-abdominal pressures on the vessels such as vena cava.

In general terms, the individual experiencing acceleration that may lead to G-LOC may engage his/her internal bodily muscle pump sufficiently to lift blood against the acceleration forces. A rhythmical application of the body's muscle pump helps to push waves of blood back towards the heart. The waves bring a bolus of blood from one venous valve to the next utilizing the muscle pump.

In existing voluntary muscle contraction and forced exhalation/breathing techniques, individuals subjected to large enough acceleration to experience G-LOC are supplied with pressure suits that apply forces from outside of the body towards the inside of the body to reduce leg and abdominal volume at moments when excessive acceleration is being detected. This helps to minimize the filling of reservoirs (such as leg veins and abdominal vein portions) with blood and thereby allow for more blood to return to the heart. At the same time, if the pressure is applied continuously, the drawback of the pressure suit is that the blood supply to the feet and the lower body is hindered. The pressure suit relies on applied gravitational and acceleration forces to supply the blood to the feet and the lower body muscles that are supposed to be used as the muscle pump to get the blood back up on a venous return path. In other words, there is a limit to the amount of static pressure that the pressure suit can apply in order to not numb the legs or the abdomen in the process of trying to save e.g., the aircraft pilot from the G-LOC.

However, the voluntary muscle contraction and forced exhalation/breathing techniques may engage the muscle pumps within the body, but the amount of force applied via the pressure suits may be limited and provided until the outside forces prevent blood flow from entering, for example, the leg muscles and only allow blood to enter when the acceleration forces are so high that the G-LOC would be a problem.

Likewise, requiring the individual experiencing early symptoms of G-LOC to perform said rhythmic muscle contractions voluntarily distracts him/her from focusing on operating their vehicle to a certain extent. Pilots are trained to aviate, navigate, and communicate in that order, and focusing on abdominal and leg contractions does interfere with effective aviation, navigation, and communication during flight. An automated system that rhythmically engages the necessary muscle groups may be desirable to reduce the cognitive load on the operator of the vehicle such as a pilot.

There is, therefore, a need in the art for an electrically assisted muscle pump (EAMP) for rhythmic contraction of large muscle groups inside the body, especially inside the legs and within the abdomen that may be automated and may happen without a conscious effort of the individual pilot.

### SUMMARY

The present disclosure generally relates to an electrically assisted muscle pumping (EAMP) system and a method thereof

In a first aspect, the present disclosure provides an electrically assisted muscle pumping system. The electrically assisted muscle pumping system includes one or more sensors to measure an acceleration or a change of the acceleration of a user in at least one dimension. The electrically assisted muscle pumping system includes a processor operatively coupled with the one or more sensors, to register an orientation of the user based on the acceleration of the user. The electrically assisted muscle pumping system includes one or more electrodes placed on the user or integrated into a pressure suit of the user and operatively connected to one or more stimulation signal generators to generate electrical stimulation signals. The electrically assisted muscle pumping system includes one or more implanted helical wire structure electrodes placed inside the user and operatively connected to the one or more electrodes to receive the electrical stimulation signals from the one or more electrodes in a transcutaneous pathway and transmit the electrical stimulation signals to one or more nerves inside the body of the user. The one or more stimulation signal generators are operatively connected to the processor, to apply electric currents to the one or more electrodes on the outside of the body which overlay the one or more implanted helical wire structure electrodes(“Injectrodes”) that in turn receive the electrical signals transcutaneously and transmit said signals to nerves of interest inside the body that either directly stimulate muscles by efferent pathways or indirectly achieve the same by triggering reflexive contraction of the musculature of interest. The electrically assisted muscle pumping system includes a recorder connected to the one or more sensors and the one or more stimulation signal generators to capture the acceleration or the change of the acceleration of the user and initiation of the application of the electric currents to the one or more electrodes on the outside of the body that are in electrical communication with the one or more implanted helical wire structure electrodes.

In some embodiments, the one or more stimulation signal generators may be fitted to at least one of on/near one leg of the user, both legs of the user, an abdomen of the user, both the legs and the abdomen of the user, a thorax of the user, and a mid and lower back of the user.

In some embodiments, the one or more sensors may be to determine at least one of a weight of the user and the orientation of the user to drive muscle pump of the user and assist with blood pressure based on the acceleration of the user.

In some embodiments, the electrically assisted muscle pumping system may further include an electrical measurement (EMG) band or a mechanical band to measure a circumference of a thorax of the user and capture information when the user inhales and exhales. The processor may be to process the captured information to measure fast and forced exhalation of the user and trigger the one or more stimulation signal generators.

In some embodiments, the one or more stimulation signal generators may be to automatically engage muscles that pump venous blood against centrifugal or gravitational forces from deep inside legs and abdomen of the user and move the venous blood back towards the heart of the user based on the acceleration of the user.

In some embodiments, the one or more stimulation signal generators may be to determine electrical waveforms of the electric currents that allow for a variable contraction of muscles of the user, and send pulsatile application of the electrical waveforms to the one or more electrodes.

In some embodiments, the electrical waveforms may include at least one of: signals of 20 Hz, signals of 50 Hz, burst signals of 200 to 300 Hz, signals up to 1.5 kHz, and signals up to 100 kHz or more.

In some embodiments, the one or more sensors may be selected from any one or a combination of an audio sensor, an electromyography (EMG) sensor, and a stretch sensor to be placed in a cockpit or the pressure suit of the user, such that the user may provide an audio command to activate or modify the pulsatile application of the electrical waveforms.

In some embodiments, the one or more stimulation signal generators may be to drive the pulsatile application of the electrical waveforms in one or more locations of the body of the user in response to sensing a reduction in acceleration forces by the one or more sensors.

In some embodiments, the pulsatile application of the electrical waveforms may be varied with changing Gravitational forces that cause the user to accelerate.

In some embodiments, the system may further include an external driver that is placed into a pocket of the pressure suit and connected to one or more cables woven within the pressure suit.

In some embodiments, the one or more implanted helical wire structure electrodes may be injected to be in close proximity to the one or more nerves that are connected to muscles of the user using one or more reflexive afferent nerve pathways or directly driving muscle activity via efferent nerves to pump venous blood against centrifugal or gravitational forces.

In some embodiments, the system may be integrated into the pressure suit of the user or coupled to a transporting device via a data link or an electrical or electromagnetic coupling.

In some embodiments, the one or more electrodes placed on the user may include a radiopaque element made of a metal or a wire on an outside edge of a face of the one or more electrodes.

In some embodiments, activation of the system is triggered by a manual input instead of a measured change in the acceleration of the user, to allow the user to initiate a controlled increase in blood volume and venal blood pressure to temporarily treat hypotensive effects.

In some embodiments, the system is used within the transporting device to prevent gravity-induced loss of consciousness.

In some embodiments, the system stabilizes the user having low blood pressure and ensures sufficient perfusion of the brain and inner organs of the user.

In some embodiments, the system provides the user with muscle activation in legs and improved perfusion of the abdomen and the legs to combat at least one of restless leg syndrome, diabetic peripheral neuropathy, cold legs and/or hands, and nociceptive pain resulting from insufficient perfusion of peripheral elements of the body of the user.

In some embodiments, the system is used within the transporting device to automate an ability of the user to activate muscle pumps utilizing an augmentative device and reduce cognitive load on the user.

In a second aspect, the present disclosure provides a method for electrically assisted muscle pumping of blood. The method includes detecting, by an electrically assisted muscle pumping system, an acceleration or a change in the acceleration of a user in at least one dimension, determining, by the electrically assisted muscle pumping system, electrical waveforms of electric currents that allow for a variable contraction of muscles of the user, to be applied to one or more electrodes and one or more nerves of the user based on the detection, wherein the one or more electrodes are placed on the user or integrated into a pressure suit of the user, applying, by the electrically assisted muscle pumping system, the electric currents to the one or more electrodes and the one or more nerves of the user based on the determination, and automatically driving, by the electrically assisted muscle pumping system, pulsatile application of pressure to blood vessels in one or more locations of a body of the user to pump blood based on the applied electric currents.

Various objects, features, aspects, and advantages of the inventive subject matter will become more apparent from the following detailed description of preferred embodiments, along with the accompanying drawing figures in which like numerals represent like components.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

FIG. 1 illustrates an example representation of an electrically assisted muscle pumping system associated with a user and a transporting device, according to an embodiment of the present disclosure;

FIG. 2 illustrates an example block diagram of the electrically assisted muscle pumping system of FIG. 1, according to an embodiment of the present disclosure;

FIG. 3A illustrates an example representation of an internal device of the electrically assisted muscle pumping system of FIG. 1, according to an embodiment of the present disclosure;

FIG. 3B illustrates an example representation of nerves to which the internal device of FIG. 3A is injected, according to an embodiment of the present disclosure;

FIG. 3C illustrates an example representation of muscles surrounding veins to which the internal device of FIG. 3A is injected, according to an embodiment of the present disclosure;

FIG. 4 illustrates a flow chart of an example method for electrically assisted muscle pumping of blood, according to an embodiment of the present disclosure;

FIGS. 5A-5D illustrate example representations of the electrically assisted muscle pumping system of FIG. 1 placed at different locations of a user, according to an embodiment of the present disclosure; and

FIG. 6 illustrates an example representation of a pressure suit with placement locations of one or more electrodes, according to an embodiment of the present disclosure.

### DETAILED DESCRIPTION

The following is a detailed description of embodiments of the disclosure depicted in the accompanying drawings. The embodiments are in such details as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

In a first aspect, the present disclosure provides an electrically assisted muscle pumping system. The electrically assisted muscle pumping system includes one or more sensors to measure an acceleration or a change in the acceleration of a user in at least one dimension. The electrically assisted muscle pumping system includes a processor operatively coupled with the one or more sensors, and provided to register an orientation of the user based on the acceleration of the user. The electrically assisted muscle pumping system includes one or more electrodes placed on the user or integrated into a pressure suit of the user and operatively connected to one or more stimulation signal generators to generate electrical stimulation signals. The electrically assisted muscle pumping system includes one or more implanted helical wire structure electrodes placed inside the user and operatively connected to the one or more electrodes to receive the electrical stimulation signals from the one or more electrodes in a transcutaneous pathway and transmit the electrical stimulation signals to one or more nerves inside the body of the user. The one or more stimulation signal generators are connected to the processor and adapted to apply electric currents to the one or more electrodes. The electrically assisted muscle pumping system includes a recorder connected to the one or more sensors and the one or more stimulation signal generators to capture the acceleration or the change in the acceleration of the user and initiation of the application of the electric currents applied to the one or more electrodes on the outside of the body that are in electrical communication with the one or more implanted helical wire structure electrodes.

In a second aspect, the present disclosure provides a method for electrically assisted muscle pumping of blood. The method includes detecting, by the electrically assisted muscle pumping system, an acceleration or a change in the acceleration of a user in at least one dimension, determining, by the electrically assisted muscle pumping system, electrical waveforms of electric currents that allow for a variable contraction of muscles of the user, to be applied to one or more electrodes and one or more nerves of the user based on the detection, wherein the one or more electrodes are placed on the user or integrated into a pressure suit of the user, applying, by the electrically assisted muscle pumping system, the electric currents to the one or more electrodes and the one or more nerves of the user based on the determination, and automatically driving, by the electrically assisted muscle pumping system, pulsatile application of pressure to blood vessels in one or more locations of a body of the user to pump blood based on the applied electric currents.

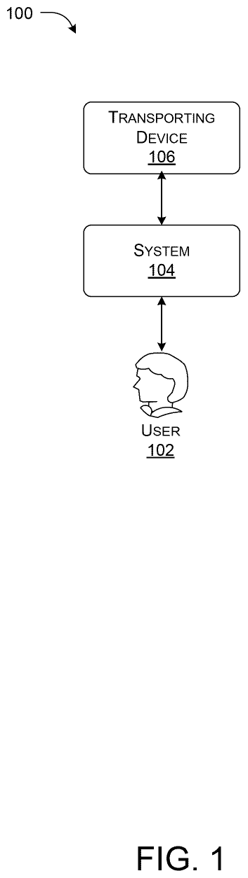

Various embodiments of the present disclosure will be explained in detail with reference to FIGS. 1-6.

FIG. 1 illustrates an exemplary representation 100 of an electrically assisted muscle pumping system associated with a user and a transporting device, according to an embodiment of the present disclosure.

Referring to FIG. 1, the electrically assisted muscle pumping system 104 may be in communication with a user 102, for example, a pilot. In some embodiments, the electrically assisted muscle pumping system 104 may be coupled to a transporting device 106 via a data link and or other forms of an electrical or electromagnetic coupling. Examples of the transporting device 106 may include, but not limited to, an aircraft, an airplane, a watercraft, and a land craft. The electrically assisted muscle pumping system 104 is used within the transporting device 106 to prevent gravity-induced loss of consciousness of the user 102. The electrically assisted muscle pumping system 104 is used within the transporting device 106 to automate the ability of the user 102 to activate muscle pumps utilizing an augmentative device and reduce cognitive load on the user 102, for example, the pilot. In some embodiments, the electrically assisted muscle pumping system 104 may be coupled, for example, in/on a chair or a seat of the transporting device 106. It may be appreciated that the electrically assister muscle pumping system 104 may be interchangeably referred as the system 104 throughout the disclosure.

In some embodiments, the electrically assisted muscle pumping system 104 may be integrated into or within a pressure suit of the user 102 to allow for a rhythmic contraction of large muscle groups inside a body of the user 102, especially inside legs and within the abdomen of the user 102. The pressure suits may be for combating Gravity-induced Loss Of Consciousness (G-LOC) which apply more or less static pressure to the legs and the abdomen of the user 102. The rhythmic contraction of the large muscle groups inside the body of the user 102 is automated and enabled without a conscious effort of the user 102 using the electrically assisted muscle pumping system 104. Therefore, the electrically assisted muscle pumping system 104 may be provided to combat the G-LOC of the user 102. In some other embodiments, the electrically assisted muscle pumping system 104 may capture an augmentation of human performance by modulating the blood pressure of the user 102. The electrically assisted muscle pumping system 104 may help individuals (e.g., the user 102) with low blood pressure to treat their hypotension. The electrically assisted muscle pumping system 104 may regulate blood pressure backup in hypertensive situations. In some other embodiments, the electrically assisted muscle pumping system 104 may be utilized in an emergency medical unit in a trauma setting where an individual has lost blood, goes into shock, or is in need of increasing blood pressure acutely or chronically.

In some embodiments, the electrically assisted muscle pumping system 104 may be triggered by a manual input, for example, a hand instead of a measured change in the acceleration of the user, and then operated for a certain duration of time. The electrically assisted muscle pumping system 104 may be activated to allow the user 102 to initiate a controlled increase in blood volume and, if desired, venal blood pressure to temporarily treat hypotensive effects. In some embodiments, the electrically assisted muscle pumping system 104 may provide the user 102 with muscle activation in the legs and improved perfusion of the abdomen and the legs to combat at least one of restless leg syndrome, diabetic peripheral neuropathy, cold legs and/or hands, and nociceptive pain resulting from insufficient perfusion of peripheral elements of the body, such as, feet, toes, hands, and fingers of the user 102.

In some embodiments, electrical currents may have to be applied to nerves innervating the large muscle groups of the legs and the abdomen of the user 102, for example, via branches of a sciatic nerve or intercostal nerves or to an anterolateral abdominal wall including thoracoabdominal, lateral cutaneous, subcostal, iliohypogastric, and ilioinguinal nerves for the rhythmic contraction of large muscle groups. The thoracoabdominal nerves are derived from T7-T11 nerves and form inferior intercostal nerves. These nerves run along the internal obliques and the transversalis muscles and are electrically not accessible from a surface of a skin of the user 102 without applying electrical fields that may be likely painful and potentially dangerous to the user 102. Therefore, the electrically assisted muscle pumping system 104 may be utilized to gain electrical access to the nerves with an ability to electrically place, for example, a transcutaneous electrical nerve stimulator (TENS) electrode on the outside of the body of the user 102. The electrically assisted muscle pumping system 104 may transcutaneously transfer sufficient current across the skin to engage deep tissue nerves that innervate major muscle groups within the body of the user 102.

Further, the electrically assisted muscle pumping system 104 may sense an acceleration or a change in the acceleration of the body of the user 102 and apply sufficient current to one or more TENS electrodes or augmented TENS (aTENS) electrodes placed on the outside of the body of the user 104. The electrically assisted muscle pumping system 104 may aid the individual user 102 with a more intense muscle pump, and rely less on static pressures applied by the pressure suit. The electrically assisted muscle pumping system 104 provides a more comfortable and a less distracting flying experience. The electrically assisted muscle pumping system 104 may allow the user 102 to shift more concentration to flying (aviate, navigate, communicate, etc.) as the electrically assisted muscle pumping system 104 automatically applies pressures when needed, reduces cognitive load, and frees up cortical resources.

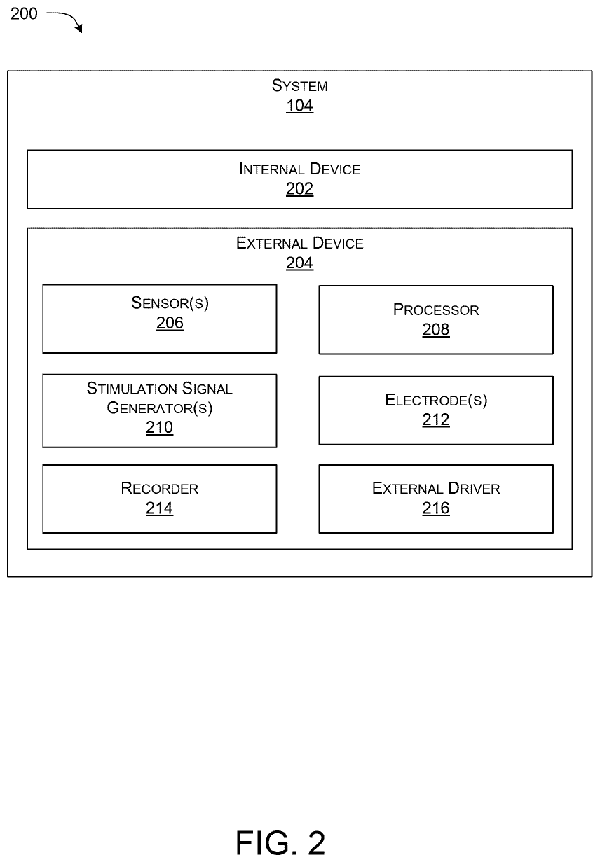

FIG. 2 illustrates an exemplary block diagram 200 of the electrically assisted muscle pumping system 104 of FIG. 1, according to an embodiment of the present disclosure.

Referring to FIG. 2, the electrically assisted muscle pumping system 104 may include an internal device 202 and an external device 204. In some embodiments, the internal device 202 may include a minimally invasive injectable component. The minimally invasive injectable component may include injectable or implantable wire structure electrodes such as, but not limited to, helical wire structure electrodes. The helical wire structure electrodes may be injected to be in close proximity to one or more nerves that are connected to muscles of the user 102 using one or more reflexive afferent nerve pathways or directly driving muscle activity via efferent nerves to pump venous blood against centrifugal or gravitational forces.

In some embodiments, the internal device 202 (e.g., helical wire structure electrodes) may act as a conduit. The internal device 202 may pick up electrical signals from just below the skin (subcutaneous tissue) of a user (e.g., 102) and transmit the electrical signals (by having a low electrical impedance) deeper into the body of the user 102. Thus, the internal device 202 enables electrical stimulation of nerves that then connect to large muscle groups for muscle pump.

In some embodiments, the external device 204 may be combined with or integrated into the pressure suit of the user 102. Referring to FIG. 2, the external device 204 may include one or more sensors 206, a processor 208, one or more stimulation signal generators 210, one or more electrodes 212, a recorder 214, and an external driver 216. The one or more sensors 206, the processor 208, the stimulation signal generator 210, the one or more electrodes 212, the recorder 214, and the external driver 216 may be operatively connected with each other.

In some embodiments, the external device 204 includes the one or more sensors 206 capturing a breathing of the user 102. The one or more sensors 206 are adapted to measure an acceleration or a change in the acceleration of a user 102 in at least one dimension, and preferentially in all three dimensions. The one or more sensors 206 are adapted to determine a height of the user 102, a weight of the user 102, and an orientation of the user 102 based on the acceleration of the user. In some embodiments, the one or more sensors 206 may include, but not limited to, an audio sensor, an electromyography (EMG) sensor, and a stretch sensor to be placed in a cockpit or the pressure suit of the user 102, such that the user 102 provides an audio command or other commands to activate or modify pulsatile application of electrical waveforms. This may allow the user 102 to disengage or reengage the system 104 on demand in a simple and faster manner.

In some embodiments, the external device 204 may include the processor 208. The processor 208 may be implemented as one or more microprocessors, microcomputers, microcontrollers, digital signal processors, central processing units, logic circuitries, and/or any devices that manipulate data based on operational instructions. Among other capabilities, the processor 208 may be configured to fetch and execute computer-readable instructions stored in the recorder 214 of the electrically assisted muscle pumping system 104. The processor 208 may be coupled with the one or more sensors 206. The processor 208 is provided to register the orientation of the user determined based on the acceleration of the user.

In some embodiments, the one or more electrodes 212 are inserted on the user 102 or integrated into a pressure suit of the user 102. The one or more electrodes 212 may be, for example, hydrogel electrodes, transcutaneous electrical nerve stimulator (TENS) electrodes, augmented TENS (aTENS) electrodes, or carbon or metal disks. The pressure suit may include terminal electrode locations which may be in specific locations that are very close to and expected to overlap the minimally invasive injectable component (e.g., helical wire structure electrodes) placed in the user 102. The pressure suit may include movable or immovable electrode locations on the inside facing the legs and/or the abdomen or the back of the user 102. The one or more electrodes 212 may be operatively connected to the one or more stimulation signal generators 210, for example, using leads to generate electrical stimulation signals. The one or more stimulation signal generators 210 may be connected to the processor 208 and adapted to apply electric currents to the one or more electrodes 212.

In some embodiments, the internal device 202 placed completely inside the user 102 may receive electrical stimulation signals from the externally placed one or more electrodes 212 in a transcutaneous pathway. Further, the internal device 202 may transmit the electrical stimulation signals to one or more nerves inside the body to drive muscle activity.

In some embodiments, an electrode patch may include a metal ring on the outside of the one or more electrodes 212, or the one or more electrodes 212 placed on the user may include a radiopaque element, for example, a ring made of a metal or a wire on the outside edge of a face of the one or more electrodes 212. The metal ring may be used to visualize the edge(s) of the one or more electrodes 212 on X-ray and to make the alignment of the electrode patches perfect. In some embodiments, the electrode patch may be a rectangular patch with rounded corners underlying the one or more stimulation signal generators 210. Since the metal ring is included on the outside of the one or more electrodes 212, the one or more electrodes 212 may be radio-opaque and easy to be utilized in an X-ray setting for an initial fitting of the one or more electrodes 212 into an undergarment or the pressure suit itself. The one or more electrodes 212 may be fitted to the user 102 or the undergarment, or the pressure suit based on where the internal device 202 have been placed prior. The fitting of the one or more electrodes 212 may be done under X-ray setting. It may be appreciated that X-ray or fluoroscopy may be used to perfectly overlay the one or more electrodes 212 over the location of the subcutaneous end of the internal device 202.

In some embodiments, the one or more stimulation signal generators 210 may be integrated onto the pressure suit of the user 102. The one or more stimulation signal generators 210 may be adapted to be fitted to at least one of, but not limited to, on/near one leg of the user 102, both legs of the user 102, an abdomen of the user 102, both the legs and the abdomen of the user 102, a thorax of the user 102, and a mid and lower back of the user 102. The one or more stimulation signal generators 210 are adapted to automatically engage muscles that pump venous blood against centrifugal or gravitational forces from deep inside the legs and the abdomen of the user 102 and push/move the venous blood back towards a heart of the user 102 based on the acceleration of the user 102.

In some embodiments, the one or more stimulation signal generators 210 are configured to determine electrical waveforms of the electric currents that allow for a variable contraction of the muscles of the user 102. In some embodiments, the one or more stimulation signal generators 210 provide charge-balanced electrical waveforms in a typical TENS frequency and a pulse width spectrum to the one or more electrodes 212. The one or more stimulation signal generators 210 send the application of the electrical waveforms to the one or more electrodes 212 which overlay a collector portion of the minimally invasive injectable component. The application of the electrical waveforms may be in 1 second on, 1 second off, or 0.5 sec on, 0.5 sec off; or 0.5sec on, 1 sec off; or 1 sec on, 0.5 sec off; or 2 sec on, 1 sec off; or 1 sec on, 2 sec off; or 0.25 sec on, 0.25 sec off; or 0.25 sec on, 0.5 sec off; or 0.5 sec on, 0.25 sec off; or other similar combinations of times ranging from a selection of times from 0.1 seconds to 10 seconds. Different muscle groups may receive different pulsing timing and duration.

In some embodiments, the electrical waveforms may include, but not be limited to, signals of 20 Hz, signals of 50 Hz, burst signals of 200 to 300 Hz, signals up to 1.5 kHz, and signals up to 100 kHz or more. The pulsatile application of the electrical waveforms is varied with changing Gravitational forces (G-forces) that cause the user 102 to accelerate.

In some embodiments, the one or more stimulation signal generators 210 are adapted to drive pulsatile application of the electrical waveforms in one or more locations of the body of the user 102 when the one or more sensors 206 sense a reduction in acceleration forces.

In some embodiments, the recorder 214 may be also referred to as a memory to store one or more computer-readable instructions or routines, which may be fetched and executed to capture sensor signals and the electrical currents applied to the one or more electrodes 212. The recorder 214 may include any non-transitory storage device including, for example, volatile memory such as a Random-Access Memory (RAM), or a non-volatile memory such as an Erasable Programmable Read-Only Memory (EPROM), a flash memory, and the like.

The recorder 214 may be connected to the one or more sensors 206 and the one or more stimulation signal generators 210 to capture sensor signals and the electrical currents applied to the one or more electrodes 212. Further, the recorder 214 may be configured to collect feedforward and feedback information and optimize sensory trigger points in the body of the user 102, the electrical currents applied to the one or more electrodes 212, and specific flight (or drive) conditions of the user 102.

In some embodiments, the external device 204 may include the external driver 216 that is placed into a pocket of the pressure suit and connected to one or more cables woven in within the pressure suit of the user 102. The one or more cables may be a part of the pressure suit or the undergarment that is worn below the pressure suit. The undergarment may add the one or more electrodes 212 that need to be lined up with the location where the internal device 202 is placed inside the body of the user 102 so that the nerves are easily activated. The undergarment or the pressure suit may be fitted to the user 102 after the internal device 202 has been placed by, for example, a clinician. Given the nature of, for example, sweating in the undergarment or the pressure suit, the undergarment or the pressure suit may be used as a single-use device that connects to the external device 204 (e.g., kept in a separate pocket in the undergarment or the pressure suit) and provides the cables and the one or more electrodes 212 at the correct locations. The undergarment may add the functionality of the system 104 and ease the use of the system 104 for the user 102 by being a single-use or easy-to-wash system at a lower cost than the pressure suit.

In some embodiments, the external device 204 may include an electrical measurement (EMG) band or a mechanical band to measure a circumference of the thorax of the user 102 and capture information when the user 102 inhales and exhales. The processor 208 may process the captured information to measure fast and forced exhalation of the user 102 and trigger the one or more stimulation signal generators 210 fitted to lower legs and abdomen of the user 102.

Further, the system 104 may also include other units such as a display unit, an input unit, an output unit, a control unit and the like, however, the same are not shown in the FIG. 2, for the purpose of clarity. The system 104 may include multiple such units or the system 104 may include any such numbers of the units, obvious to a person skilled in the art or as required to implement the features of the present disclosure.

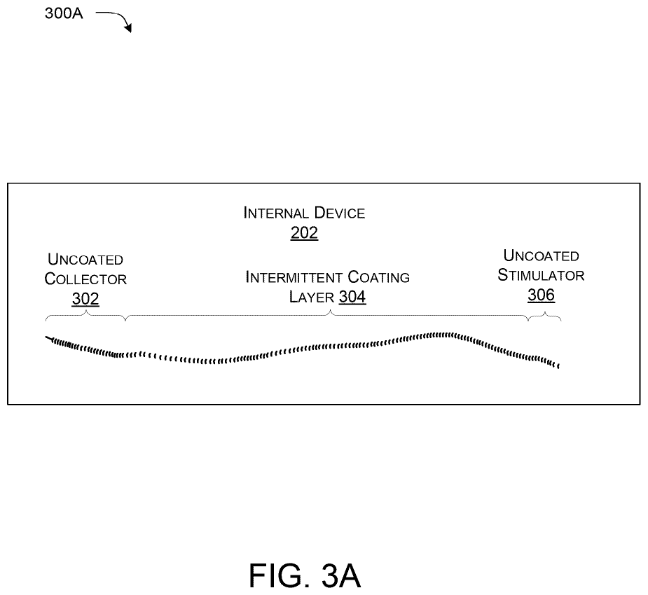

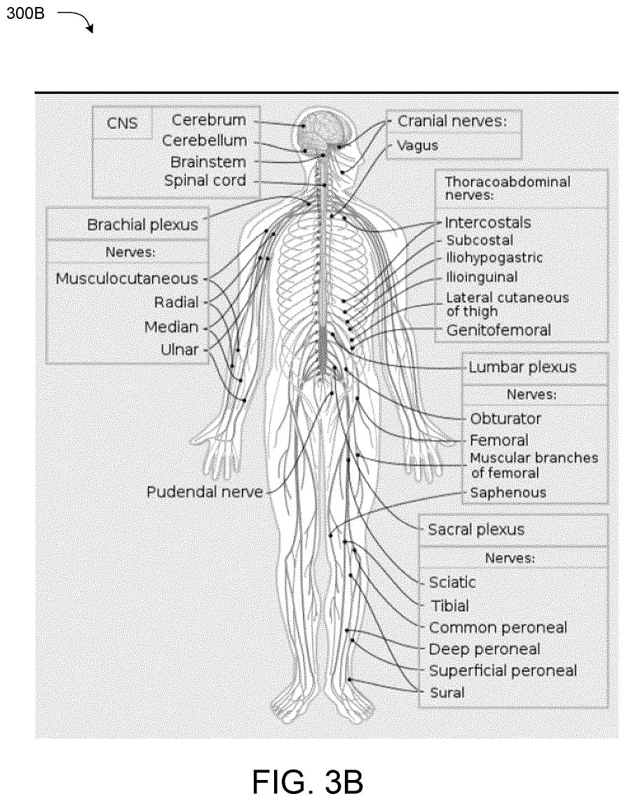

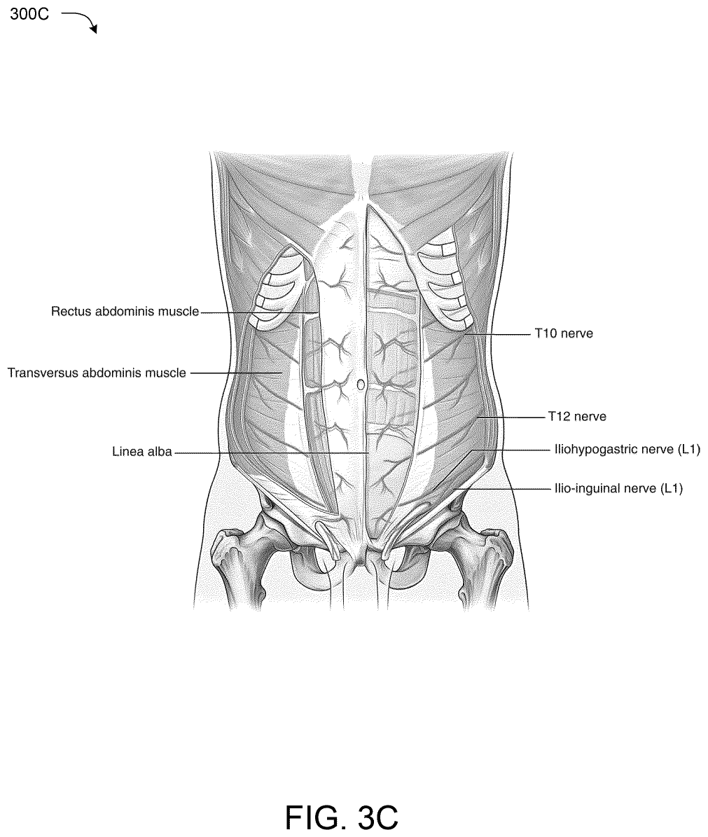

FIG. 3A illustrates an exemplary representation 300 of an internal device 202 of the electrically assisted muscle pumping system 104 of FIG. 1, according to an embodiment of the present disclosure.

Referring to FIG. 3A, the internal device 202, for example, the helical wire structure electrodes may be injected to be in close proximity to a muscle directly or indirectly utilizing reflexive pathways. The internal device 202 (e.g., helical wire structure electrodes) may act as a conduit. The internal device 202 may pick up electrical signals from just below the skin (subcutaneous tissue) of the user 102 and transmit the electrical signals (by having a low electrical impedance) deeper into the body of the user 102. Thus, the internal device 202 enables electrical stimulation of nerves that then connect to large muscle groups.

In some embodiments, the internal device 202 may engage with the user body's sensors in muscles, muscle spindles, golgi tendon organs, and lower-level processing centers in a spinal cord to drive a body's venous muscle pump, such that the blood is pumped against a force of gravity and forces mimicking and multiplying gravitational force effects via centrifugal acceleration. The body's muscle pump is utilized to generate and apply centripetal forces on the venous blood to raise/move it back towards the heart of the user 102. The internal device 202 including injectable or implantable wire structure electrode, such as the helical wire structure electrode is further described in PCT/US2021/033007. The internal device 202 may also include an uncoated collector 302, intermittent coating layer 304, and an uncoated stimulator 306.

The internal device 202 may be injected into, onto, nearby, or around the nerves of the user 102. The internal device 202 may be injected into specific nerves of interest driving either afferent input to a spinal cord or driving muscles directly to augment muscle activity in the abdomen or the legs of the user 102. The nerves of the user 102 include, without limitation, cranial nerves, thoracoabdominal nerves, lumbar plexus nerves, sacral plexus nerves, gluteal nerves, pudendal nerves, brachial plexus nerves, and the like, as shown in representation 300B of FIG. 3B.

FIG. 3C illustrates an example representation 300C of muscles surrounding veins to which the internal device 202 of FIG. 3A is injected, according to an embodiment of the present disclosure.

With respect to FIG. 3C, the internal device 202 may engage with the user body's sensors in muscles, for example, rectus abdominis muscle or transversus abdominis muscle, or the muscle spindles surrounding the blood vessels or the veins in the leg/abdomen of the user 102. The engagement of the internal device 202 with the muscles may push the blood up from, for example, the legs of the user 102 and drive the blood towards the heart of the user 102. The internal device 202 may drive the muscles by activating nerves either by efferent direct drive or by efferent reflexive drive resulting in efferent signals for muscle activation.

In some embodiments, efferent activation of the nerves for direct drive may be achieved at continuous waveform portions (e.g., up to 2 or even up to 5 seconds long pulse trains) of 20 to 50 Hz. Electric currents may be applied to efferent nerve branches (rootlets) between the spinal cord and Dorsal Root Ganglion (DRG), combined nerve portions distal to the DRG or to Peripheral Nervous System (PNS) nerve portions distal to the DRG such as the Thoracoabdominal nn. (T6-T11), Subcostal n. (T12), iliohypogastric nerve (L1), and ilioinguinal nerve (L1). Stimulation may be applied to abdominal, thorax, or leg nerves such as the sciatic or branches of the sciatic nerve.

In some embodiments, efferent activation of the nerves for reflexive drive may be achieved at continuous or intermittent waveform portions of 30 to 80 Hz. The waveform amplitude and/or pulse width may be modulated to provide a rhythmic or arrhythmic, pulsatile, or at least variable contraction of the muscles. This in turn triggers both muscle spindles and golgi tendon organs to report active and passive muscle engagement in certain instances driven by nearby muscles and muscle portions, thereby causing a reflexively mediated muscle contraction.

In some embodiments, efferent drive may be applied to the DRG of the corresponding muscle groups of interest or to the efferent dorsal rami from the spinal cord to the DRG or it may be applied to the combined nerve portions distal to the DRG or to the PNS nerve portions distal to the DRG such as the Thoracoabdominal nn. (T6-T11), Subcostal n. (T12), iliohypogastric nerve (L1), ilioinguinal nerve (L1). Efferent drive is best applied in bursting or pulsatile fashion to engage the spinal cord to generate reflexively induced contractions of the corresponding muscle portions.

A stimulating end of the injected electrode e.g., the helical wire structure electrode may be placed onto/into/around/against:
a. Thoracoabdominal nn. (T6-T11) for the direct drive, efferent engagement,
b. Subcostal n. (T12) for the direct drive, efferent engagement,
c. iliohypogastric nerve (L1) for the direct drive, efferent engagement,
d. ilioinguinal nerve (L1) for the direct drive, efferent engagement,
e. Dorsal root ganglia on same (T6-L1) levels to induce a reflexive engagement, and
f. Combined nerves (T6-L1) or their efferent transforaminal component for either the direct drive or an induced “tickle of muscle spindles” reflexive drive component.

In order to retain proper alignment and stability for the user 102 in/on their chair or seat within an aircraft, a watercraft, or a land craft, stabilizing muscles may be engaged in addition to the abdominal muscles. Examples of such muscles may include, but are not limited to, obliques, gluteus maximus, and related muscle groups. Similar to the abdominal muscles and their corresponding nerves, the stabilizing muscle groups may be engaged from their nerve rootlets to the DRG to the combined nerve leaving a foramina to a specific nerve plexus and nerve branches of interest all the way into the muscles at a neuromuscular junction.

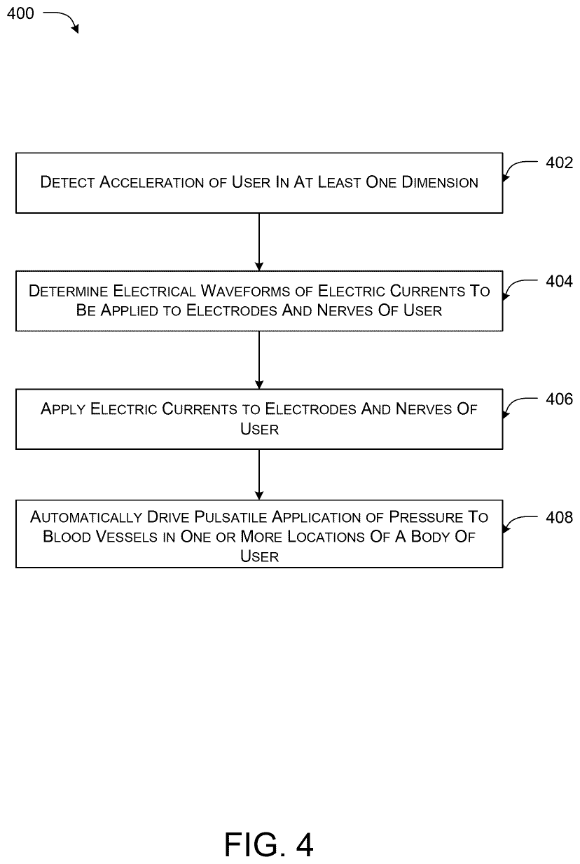

FIG. 4 illustrates a flow chart of an example method 400 for electrically assisted muscle pumping of blood, according to an embodiment of the present disclosure.

Referring to FIG. 4, at block 402, the method 400 includes detecting, by the electrically assisted muscle pumping system 104, an acceleration or a change in the acceleration of a user 102 in at least one dimension, preferentially all the three dimensions.

At block 404, the method 400 includes determining electrical waveforms of electric currents that allow for a variable contraction of the muscles of the user 102, to be applied to one or more electrodes 212 and one or more nerves of the user 102 based on the detection. The one or more electrodes 212 are placed on the user 102 or integrated into a pressure suit of the user 102.

At block 406, the method 400 includes applying the electric currents to the one or more electrodes 212 and to the one or more nerves of the user based on the determination.

At block 408, in response to the determination, the method 400 includes automatically driving pulsatile application of pressure to blood vessels in one or more locations of a body of the user 102 to pump blood based on a reduction in acceleration forces being detected and the applied electric currents.

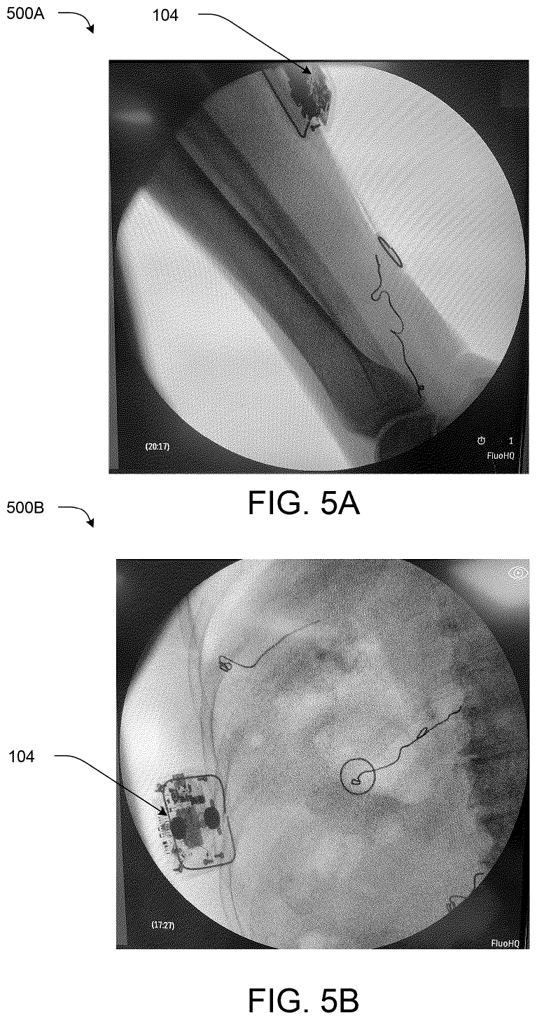

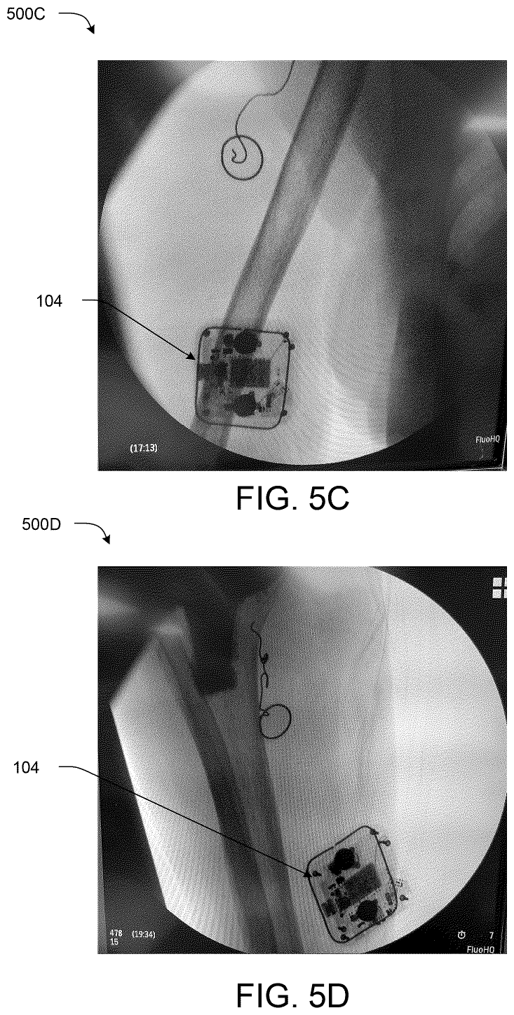

FIGS. 5A-5D illustrate example representations 500A-500D of the electrically

assisted muscle pumping system 104 of FIG. 1 placed at different locations of a user 102, according to an embodiment of the present disclosure.

With respect to FIGS. 5A-5D, the electrically assisted muscle pumping system 104 may be placed at different locations of the user 102. For example, the electrically assisted muscle pumping system 104 may be placed on/near one leg of the user 102, both legs of the user 102, the abdomen of the user 102, both the legs and the abdomen of the user 102, a thorax of the user 102, and a mid and lower back of the user 102. FIGS. 5A-5D depict that the internal device 202 is injected on at least one of the abdominal nerves, the intercostal nerves, the sciatic nerve, and branches of the sciatic nerve. The one or more stimulation signal generators 210 are placed on the outside of the body in the leg or abdominal region of the user 102 to interface electrically (transcutaneously) with the fully injected devices, for example, the internal device 202. The fully injected device 202 may be invisible to the naked eye. This means that the user 102 does not show to their surroundings (family, friends, peers) that they are utilizing the system 104 at work.

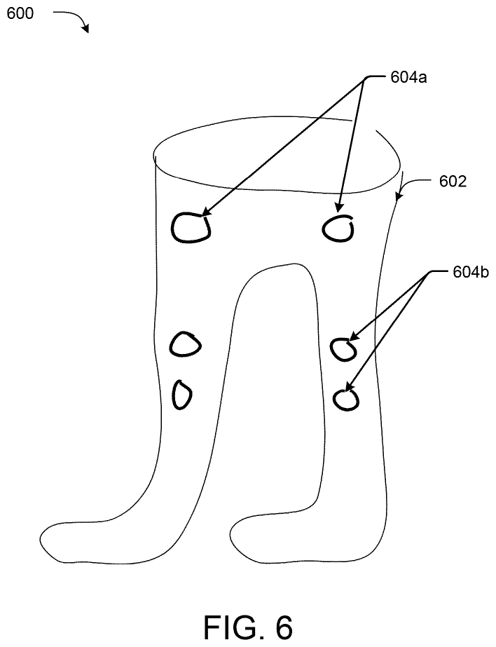

FIG. 6 illustrates an example representation 600 of a pressure suit 602 with placement locations of one or more electrodes 212, according to an embodiment of the present disclosure.

With respect to FIG. 6, the pressure suit 602 may include one or more terminal electrode locations 604a for abdominal activation, for example, upper electrode locations, and one or more terminal electrode locations 604b for leg activation. The terminal electrode locations 604a, 604b inside the pressure suit 602 may be in specific locations that are very close to and expected to overlap the internally placed injected device 202 in the user 102. The pressure suit 602 may have movable or immovable electrode locations on the inside facing the user's legs and/or abdomen or back of the user 102.

During the fitting process of the electrically assisted muscle pumping system 104, the user 102 initially receives the internal device (Injectrodes) 202 and then puts on the pressure suit 602 for X-ray imaging. The pressure suit 602 may be customed to the user 102. During the X-ray fitting procedure, one or more markers may be placed on the pressure suit 602 to enable an addition of connectors for the one or more electrodes 212 in an exact spot. In this instance, the user may take off the pressure suit 602. The one or more electrodes 212 may be added and connected to the one or more stimulation signal generators 210. Once the connectors are fully added to the pressure suit 602, the user 102 may put on the pressure suit 602 as needed knowing that the connectors with the one or more electrodes 212 are always being placed correctly over the subcutaneously placed internal device 202.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

1. An electrically assisted muscle pumping system, comprising:
one or more sensors to measure an acceleration or a change of the acceleration of a user in at least one dimension;
a processor operatively coupled with the one or more sensors, wherein the processor is to register an orientation of the user based on the acceleration of the user;
one or more electrodes placed on the user or integrated into a pressure suit of the user and operatively connected to one or more stimulation signal generators to generate electrical stimulation signals; and
one or more implanted helical wire structure electrodes placed inside the user and operatively connected to the one or more electrodes to receive the electrical stimulation signals from the one or more electrodes in a transcutaneous pathway and transmit the electrical stimulation signals to one or more nerves inside the body of the user;
the one or more stimulation signal generators operatively coupled with the processor, wherein the one or more stimulation signal generators are to apply electric currents to the one or more electrodes; and
a recorder operatively connected to the one or more sensors and the one or more stimulation signal generators to capture the acceleration or the change of the acceleration of the user and initiation of the application of the electric currents to the one or more electrodes on the outside of the body that are in electrical communication with the one or more implanted helical wire structure electrodes.

2. The system of claim 1, wherein the one or more stimulation signal generators are fitted to at least one of: on/near one leg of the user, both legs of the user, an abdomen of the user, both the legs and the abdomen of the user, a thorax of the user, and a mid and lower back of the user.

3. The system of claim 1, wherein the one or more sensors are to determine at least one of: a weight of the user and the orientation of the user to drive muscle pump of the user and assist with blood pressure based on the acceleration of the user.

4. The system of claim 1, further comprising an electrical measurement (EMG) band or a mechanical band to measure a circumference of a thorax of the user and capture information when the user inhales and exhales, wherein the processor is to process the captured information to measure fast and forced exhalation of the user and trigger the one or more stimulation signal generators.

5. The system of claim 1, wherein the one or more stimulation signal generators are to automatically engage muscles that pump venous blood against centrifugal or gravitational forces from deep inside legs and abdomen of the user and move the venous blood back towards the heart of the user based on the acceleration of the user.

6. The system of claim 1, wherein the one or more stimulation signal generators are to determine electrical waveforms of the electric currents that allow for a variable contraction of muscles of the user, and send pulsatile application of the electrical waveforms to the one or more electrodes.

7. The system of claim 6, wherein the electrical waveforms are at least one of: signals of 20 Hz, signals of 50 Hz, burst signals of 200 to 300 Hz, signals up to 1.5 kHz, and signals up to 100 kHz or more.

8. The system of claim 6, wherein the one or more sensors are selected from any one or a combination of: an audio sensor, an electromyography (EMG) sensor, and a stretch sensor to be placed in a cockpit or the pressure suit of the user, such that the user provides an audio command to activate or modify the pulsatile application of the electrical waveforms.

9. The system of claim 6, wherein the one or more stimulation signal generators are to drive the pulsatile application of the electrical waveforms in one or more locations of the body of the user in response to sensing a reduction in acceleration forces by the one or more sensors.

10. The system of claim 6, wherein the pulsatile application of the electrical waveforms is varied with changing Gravitational forces that cause the user to accelerate.

11. The system of claim 1, further comprising an external driver that is placed into a pocket of the pressure suit and connected to one or more cables woven within the pressure suit.

12. The system of claim 1, wherein the one or more implanted helical wire structure electrodes are injected to be in close proximity to the one or more nerves that are connected to muscles of the user using one or more reflexive afferent nerve pathways or directly driving muscle activity via efferent nerves to pump venous blood against centrifugal or gravitational forces.

13. The system of claim 1, wherein the system is integrated into the pressure suit of the user or coupled to a transporting device via a data link or an electrical or electromagnetic coupling.

14. The system of claim 1, wherein the one or more electrodes placed on the user comprise a radiopaque element made of a metal or a wire on an outside edge of face of the one or more electrodes.

15. The system of claim 1, wherein an activation of the system is triggered by a manual input to allow the user to initiate controlled increase in blood volume and venal blood pressure to temporarily treat hypotensive effects.

16. The system of claim 13, wherein the system is used within the transporting device to prevent gravity-induced loss of consciousness.

17. The system of claim 1, wherein the system stabilizes the user having low blood pressure and ensures sufficient perfusion of brain and inner organs of the user.

18. The system of claim 1, wherein the system provides the user with muscle activation in legs and improved perfusion of abdomen and the legs to combat at least one of: restless leg syndrome, diabetic peripheral neuropathy, cold legs and/or hands, and nociceptive pain resulting from insufficient perfusion of peripheral elements of the body of the user.

19. The system of claim 16, wherein the system is used within the transporting device to automate an ability of the user to activate muscle pumps utilizing an augmentative device and reduce cognitive load on the user.

20. A method for electrically assisted muscle pumping of blood, comprising:
detecting, by an electrically assisted muscle pumping system, an acceleration or a change in the acceleration of a user in at least one dimension;
determining, by the electrically assisted muscle pumping system, electrical waveforms of electric currents that allow for a variable contraction of muscles of the user, to be applied to one or more electrodes and one or more nerves of the user based on the detection, wherein the one or more electrodes are placed on the user or integrated into a pressure suit of the user;
applying, by the electrically assisted muscle pumping system, the electric currents to the one or more electrodes and the one or more nerves of the user based on the determination; and
automatically driving, by the electrically assisted muscle pumping system, pulsatile application of pressure to blood vessels in one or more locations of a body of the user to pump blood based on the applied electric currents.
